(19) United States
(12) Patent Application Publication
(10) Pub. No.: US 2018/0160911 A1
(43) Pub. Date: Jun. 14, 2018

(54) Physiological Monitoring Sensor Strip and Method for Manufacturing the Same, Physiological Monitoring Mattress and Physiological Monitoring System Including the Same

(71) Applicant: Nazhiyuan Technology (Tangshan), LLC., Tangshan (CN)

(72) Inventors: Xiaoyue FU, Tangshan (CN); Shan WANG, Tangshan (CN); Shun FENG, Tangshan (CN); Qiang ZHONG, Tangshan (CN); Xiaoxiong WANG, Tangshan (CN); Hao ZHAO, Tangshan (CN); Nam MIU, Tangshan (CN); Charles HSU, Tangshan (CN)

(21) Appl. No.: 15/119,663

(22) PCT Filed: Dec. 3, 2015
(86) PCT No.: PCT/CN2015/096344

§ 371 Date: Aug. 17, 2016

(30) Foreign Application Priority Data
Nov. 23, 2015 (CN) 201510814916.5

(51) Int. Cl. A61B 5/0205; A61B 5/113

(52) U.S. Cl. CPC A61B 5/0205; A61B 5/0006; A61B 5/113

(57) ABSTRACT

Disclosure includes a physiological monitoring sensor strip and a method for manufacturing the same, and a physiological monitoring mattress and a physiological monitoring system having the same. The physiological monitoring sensor strip includes: a first triboelectric layer and a second triboelectric layer in a stacked arrangement, between which a triboelectric interface is formed; an insulation layer wrapping the first triboelectric layer and wrapping the second triboelectric layer; an electric conduction shielding layer wrapping the first triboelectric layer, the second triboelectric layer, and the insulation layer; and a first extraction electrode and a second extraction electrode acting as outputting electrodes of the physiological monitoring sensor strip.

### FIELD

The present disclosure generally relates to the field of medical and physiological equipment and technology, and more particularly to a physiological monitoring sensor strip and a method for manufacturing the same, a physiological monitoring mattress and a physiological monitoring system including the same.

### BACKGROUND

Physical indicators, such as breath and heartbeat, are of great significance for monitoring human health and diagnosing and treating disease, particularly for patients. However, there are many problems exist in the existing instruments for monitoring physical indicators, such as breath and heartbeat.

Most existing respiratory monitoring equipment is expensive and complicated, and needs to be operated by a professional. In addition, individual is required to wear a nose-mouth type monitor when monitored, which is likely to cause discomfort and psychological pressure to individual being monitored.

Most existing heartbeat monitoring equipment is electrocardiograph (ECG) which commonly is very expensive and requests an operator to be professional trained. Moreover, individual is required to paste a plurality of lead electrodes when monitored, which leads an upper body of the individual to be covered with connected wires, which is likely to cause a great psychological pressure to individual, and thus interferes with diagnostic results. In addition, ECG signal also is susceptible to external electromagnetic interference. Further, when the individual is sleeping, the lead electrodes pasted on the body of the individual may fall off as the individual changes his sleeping position, which eventually lead to a failure of the whole monitoring, and thus unable to achieve the purpose of monitoring.

In addition, most existing instruments for monitoring physical indicators, such as breath and heartbeat, are used independently for monitoring a special item. For example, the existing electrocardiograph (ECG) does not take a function for monitoring breath into consideration, the existing respiratory monitoring equipment does not take a function for monitoring heartbeat into consideration, either, which makes it difficult to real-timely and comprehensively monitor the health condition of the individual being monitored.

In summary, the existing instruments for monitoring physical indicators, such as breath and heartbeat, prevalently have a plurality of drawbacks, such as single function, susceptible to external interference, poor reliability, complex structure and manufacturing process, complicated operation, and high cost.

### SUMMARY

An object of the present disclosure directs to drawbacks in the related art, providing in embodiments a physiological monitoring sensor strip and a method for manufacturing the same, a physiological monitoring mattress, and a physiological monitoring system, so as to solve the problems exiting in the related art, such as single function, susceptible to external interference, poor reliability, complex structure and manufacturing process, complicated operation, and high cost.

According to a first aspect of the present disclosure, a physiological monitoring sensor strip is provided. The physiological monitoring sensor strip includes: a first triboelectric layer and a second triboelectric layer in a stacked arrangement, between which a triboelectric interface is formed; an insulation layer wrapping the first triboelectric layer and wrapping the second triboelectric layer; an electric conduction shielding layer wrapping the first triboelectric layer, the second triboelectric layer, and the insulation layer; and a first extraction electrode and a second extraction electrode acting as outputting electrodes of the physiological monitoring sensor strip, wherein the first extraction electrode and the second extraction electrode are connected to the first triboelectric layer and the second triboelectric layer, respectively; or the first extraction electrode is connected to the first triboelectric layer or the second triboelectric layer, and the second extraction electrode is connected to the electric conduction shielding layer.

According to a second aspect of the present disclosure, a physiological monitoring system is provided. The physiological monitoring system includes the physiological monitoring sensor strip described above, and further includes a terminal device for statistically analyzing a physiological electrical signal output by the physiological monitoring sensor strip and displaying a result of the statistical analysis.

According to a third aspect of the present disclosure, a physiological monitoring mattress is provided. The physiological monitoring mattress includes the physiological monitoring sensor strip described above and a mattress body, wherein the physiological monitoring sensor strip is disposed inside and/or outside the mattress body.

According to a fourth aspect of the present disclosure, a physiological monitoring system is provided. The physiological monitoring system includes the physiological monitoring mattress described above, and further includes a terminal device for statistically analyzing a physiological electrical signal output by the physiological monitoring mattress and displaying a result of the statistical analysis.

According to a fifth aspect of the present disclosure, a method for manufacturing a physiological monitoring sensor strip is provided. The method includes: manufacturing a first triboelectric layer and a second triboelectric layer, and disposing the first triboelectric layer and the second triboelectric layer in a stacked arrangement by which a triboelectric interface is formed therebetween; tailoring an insulation layer such that the first triboelectric layer and the second triboelectric layer are wrapped with the insulation layer; and tailoring an electric conduction shielding layer such that the first triboelectric layer, the second triboelectric layer and the insulation layer are wrapped with the electric conduction shielding layer, wherein a first extraction electrode and a second extraction electrode are connected to the first triboelectric layer and the second triboelectric layer, respectively, prior to tailoring the insulation layer; and exposed out when wrapped with the insulation layer, or the first extraction electrode is connected to the first triboelectric layer or the second triboelectric layer prior to tailoring the insulation layer; and exposed out when wrapped with the insulation layer, and the second extraction electrode is connected to the electric conduction shielding layer prior to or subsequent to wrapped with the electric conduction shielding layer.

The physiological monitoring sensor strip and the method for manufacturing the same, the physiological monitoring mattress, and the physiological monitoring system of the present disclosure are based on a principle of triboelectric. The physiological monitoring sensor strip, the physiological monitoring mattress, and the physiological monitoring system of the present disclosure not only may be used to monitor frequencies and waveforms of human breath and heartbeat, but also to simultaneously monitor action signals (such as, turning over, leaving the bed, snoring and so on) and/or time points corresponding to action signals. Take monitoring sleep as an example, when a person is in a quiet sleep, physiological electric signals (such as voltage signals) output by the physiological monitoring sensor strip may reflect the frequencies and/or waveforms of breath and heartbeat of the person in the quiet sleep; when the person turns over, leaves the bed, or snores in the sleep, the physiological monitoring sensor strip may output physiological electric signals significantly different from that output when the person is in the quiet sleep. All of those provide an accurate and reliable data base for analyzing a sleeping quality and health condition of human body.

Comparing to the related art, the physiological monitoring sensor strip and the method for manufacturing the same, the physiological monitoring mattress, and the physiological monitoring system of the present disclosure can have one or more of the following advantages:

(1) As the physiological monitoring sensor strip, the physiological monitoring mattress, and the physiological monitoring system of the present disclosure are manufactured based on the principle of triboelectric generator construction, so they can have the characteristics of self-power supply, high sensitivity, stable output electric signals, and simple operation.

(2) The physiological monitoring sensor strip, the physiological monitoring mattress, and the physiological monitoring system of the present disclosure can achieve functions of self-moistureproof and self-shielding by disposing the insulation layer and the electric conduction shielding layer into the structure itself, which can not only increase the stability of electric signals output, but also prolong the service life.

(3) As the physiological monitoring sensor strip, the physiological monitoring mattress, and the physiological monitoring system of the present disclosure are made of an easy-cutting and flexible film material, so they can have adjustable sizes and light weights, and are comfort and convenient to use for a user.

(4) The physiological monitoring sensor strip, the physiological monitoring mattress, and the physiological monitoring system of the present disclosure can have low cost, simple structures and manufacturing processes, and are suitable for large-scale industrial manufacture.

### BRIEF DESCRIPTION OF THE DRAWINGS

FIG. 1 is a schematic view showing a cross-sectional structure of a physiological monitoring sensor strip according to embodiment 1 of the present disclosure;

FIG. 2 is a schematic view showing a cross-sectional structure of a physiological monitoring sensor strip according to embodiment 2 of the present disclosure;

FIG. 3 is a schematic view showing a cross-sectional structure of a physiological monitoring sensor strip according to embodiment 3 of the present disclosure;

FIG. 4 is a schematic view showing a cross-sectional structure of a physiological monitoring sensor strip according to embodiment 4 of the present disclosure;

FIG. 5 is a schematic view showing a cross-sectional structure of a physiological monitoring sensor strip according to embodiment 5 of the present disclosure;

FIG. 6 is a schematic view showing a cross-sectional structure of a physiological monitoring sensor strip according to embodiment 6 of the present disclosure;

FIG. 7 is a schematic view showing a cross-sectional structure of a physiological monitoring sensor strip according to embodiment 7 of the present disclosure;

FIG. 8 is a schematic view showing a cross-sectional structure of a physiological monitoring sensor strip according to embodiment 8 of the present disclosure;

FIG. 9 is a schematic view showing a cross-sectional structure of a physiological monitoring sensor strip according to embodiment 9 of the present disclosure;

FIG. 10 is a schematic view showing a cross-sectional structure of a physiological monitoring sensor strip according to embodiment 10 of the present disclosure;

FIG. 11 is a schematic view showing a second surface of a first macromolecule polymer insulation layer of a physiological monitoring sensor strip according to an embodiment of the present disclosure;

FIG. 12 is a flow chart showing one method for manufacturing a physiological monitoring sensor strip according to embodiments of the present disclosure;

FIG. 13 is a flow chart showing another method for manufacturing a physiological monitoring sensor strip according to embodiments of the present disclosure; and

FIG. 14 is a flow chart showing yet another method for manufacturing a physiological monitoring sensor strip according to embodiments of the present disclosure.

### DETAILED DESCRIPTION

Description will be made in detail with reference to the following specific embodiments in order to fully understand the purpose, characteristics and effects of the present disclosure, but the embodiments shall not be construed to limit the present disclosure.

A physiological monitoring sensor strip according to an embodiment of the present disclosure is manufactured based on the principle of triboelectric, and includes a first triboelectric layer, a second triboelectric layer, an insulation layer, an electric conduction shielding layer, and a first extraction electrode and a second extraction electrode acting as outputting electrodes of the physiological monitoring sensor strip. The first triboelectric layer and the second triboelectric layer are in a stacked arrangement, between which a triboelectric interface is formed. The insulation layer wraps the first triboelectric layer and the second triboelectric layer. The electric conduction shielding layer wraps the first triboelectric layer, the second triboelectric layer, and the insulation layer. The first extraction electrode and the second extraction electrode are connected to the first triboelectric layer and the second triboelectric layer, respectively; or the first extraction electrode is connected to the first triboelectric layer or the second triboelectric layer, and the second extraction electrode is connected to the electric conduction shielding layer.

The physiological monitoring sensor strip according to an embodiment of the present disclosure may be directly laid inside a mattress, on an upper surface of the mattress, on a lower surface of the mattress, or in an effective area within a certain distance from the mattress such that the physiological monitoring sensor strip may monitor human physical conditions normally and reflect human health status real-timely, without the need of being worn.

Alternatively, the physiological monitoring sensor strip according to an embodiment of the present disclosure may also include a protection layer wrapping an outer surface of the electric conduction shielding layer. The protection layer is disposed to not only protect an internal structure of the physiological monitoring sensor strip, thereby avoiding normal work of the internal structure from being affected by external environmental factors; but also to ensure the internal structure a clean environment; as well as to affix the physiological monitoring sensor strip to the mattress according to actual demands. The protection layer may be configured to be a detachable structure such that it is convenient for cleaning so as to ensure the physiological monitoring sensor strip the cleanness and sanitation.

The insulation layer may fully wrap both the first triboelectric layer and the second triboelectric layer to form a fully-wrapped structure, while the insulation layer may also fully wrap the first triboelectric layer but partially wrap the second triboelectric layer such that a partial region of the second triboelectric layer is in contact with the electric conduction shielding layer, forming a partially-wrapped structure. Structures of the physiological monitoring sensor strip will be described in detail with reference to several specific embodiments hereinafter.

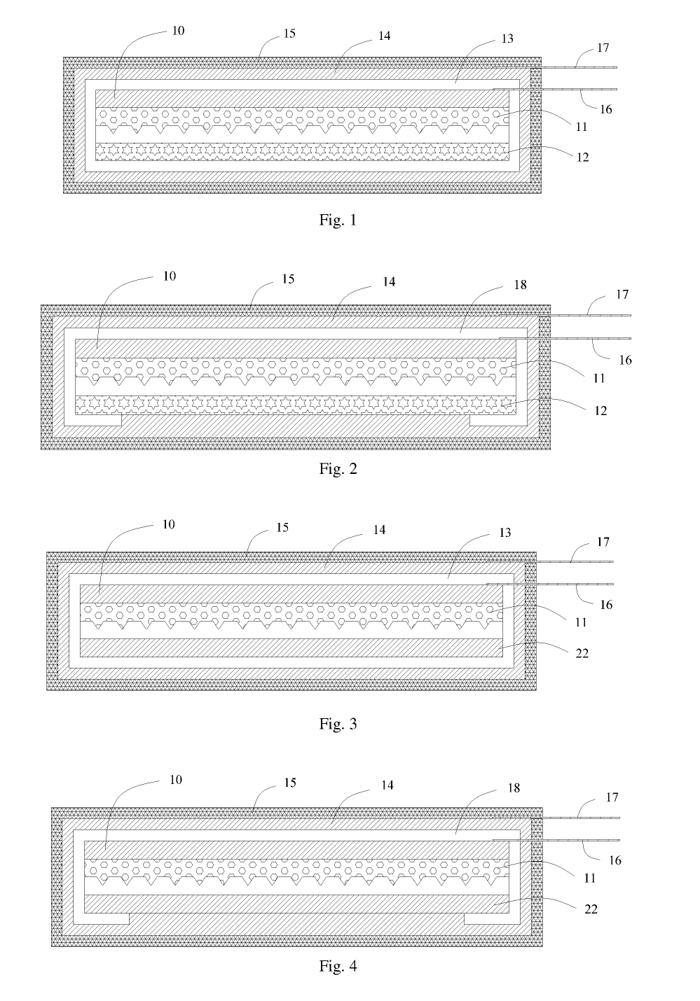

FIG. 1 is a schematic view showing a cross-sectional structure of a physiological monitoring sensor strip according to embodiment 1 of the present disclosure. In specific, FIG. 1 shows a cross-sectional schematic of respective layered structure inside the physiological monitoring sensor strip. In specific, as shown in FIG. 1, the first triboelectric layer is a first macromolecule polymer insulation layer 11 provided with a first electrode 10 on a first surface thereof, the second triboelectric layer is a second macromolecule polymer insulation layer 12, the first macromolecule polymer insulation layer 11 provided with the first electrode 10 on the first surface thereof and the second macromolecule polymer insulation layer 12 are in a stacked arrangement, and a triboelectric interface is formed between a second surface of the first macromolecule polymer insulation layer 11 not provided with the first electrode 10 thereon and the second macromolecule polymer insulation layer 12. The insulation layer 13 fully wraps the first electrode 10, the first macromolecule polymer insulation layer 11, and the second macromolecule polymer insulation layer 12. The electric conduction shielding layer 14 fully wraps an outer surface of the insulation layer 13, i.e. the electric conduction shielding layer 14 fully wraps the first electrode 10, the first macromolecule polymer insulation layer 11, the second macromolecule polymer insulation layer 12, and the insulation layer 13. The protection layer 15 fully wraps an outer surface of the electric conduction shielding layer 14, i.e. the protection layer 15 fully wraps the first electrode 10, the first macromolecule polymer insulation layer 11, the second macromolecule polymer insulation layer 12, the insulation layer 13, and the electric conduction shielding layer 14. The first electrode 10 is connected to a first extraction electrode 16, the electric conduction shielding layer 14 is connected to a second extraction electrode 17, and the first extraction electrode 16 and the second extraction electrode 17 act as outputting electrodes of the physiological monitoring sensor strip. The physiological monitoring sensor strip shown in FIG. 1 is configured to be of a fully-wrapped structure.

The insulation layer 13 is a single-side adhesive or double-side adhesive insulation tape, such as a polyethylene terephthalate tape, i.e. a PET tape, the insulation tape fully wraps the first electrode 10, the first macromolecule polymer insulation layer 11, and the second macromolecule polymer insulation layer 1 2 to form a sealed-structure, i.e. a fully-wrapped structure. The insulation layer 13 wraps the first electrode 10, the first macromolecule polymer insulation layer 11, and the second macromolecule polymer insulation layer 12, so as on one hand to play functions of waterproof and moistureproof, and thus omit a process of moistureproof treatment on the outermost surface, on the other hand to achieve package of the first electrode 10, the first macromolecule polymer insulation layer 11, and the second macromolecule polymer insulation layer 12, and thus avoid the triboelectric electricity generation of the first macromolecule polymer insulation layer 11 and the second macromolecule polymer insulation layer 12 from being affected by the external environmental factors.

The electric conduction shielding layer 14 is a single-side adhesive or non-adhesive conductive tape, and fully wraps the outer surface of the insulation layer 13. In the case that the insulation layer 13 is the single-side adhesive insulation tape, then the electric conduction shielding layer 14 is the single-side adhesive conductive tape. In specific, an adhesive-side of the insulation layer 13 is attached to the first electrode 10, the first macromolecule polymer insulation layer 11, and the second macromolecule polymer insulation layer 12, an adhesive-side of the electric conduction shielding layer 14 is attached to the outer surface of the insulation layer 13. In the case that the insulation layer 13 is the double-side adhesive insulation tape, then the electric conduction shielding layer 14 is the non-adhesive conductive tape. In specific, a first adhesive-side of the insulation layer 13 (i.e. an inter surface of the insulation layer 13) is attached to the first electrode 10, the first macromolecule polymer insulation layer 11, and the second macromolecule polymer insulation layer 12, and the electric conduction shielding layer 14 is attached to a second adhesive-side of the insulation layer 13 (i.e. the outer surface of the insulation layer 13).

The protection layer 15 may be a fabric or plastic layer, or a plastic film. In the case that the protection layer 15 is the plastic layer, then the protection layer 15 is made of thin plastic. The protection layer 15 is configured to not only protect the internal structure (the first electrode 10, the first macromolecule polymer insulation layer 11, the second macromolecule polymer insulation layer 12, the insulation layer 13, and the electric conduction shielding layer 14) of the physiological monitoring sensor strip, thereby avoiding normal work of the internal structure from being affected by external environmental factors; but also to ensure the internal structure a clean environment; as well as to fix the physiological monitoring sensor strip to the mattress according to actual demands. Preferably, the protection layer 15 may be configured to be a detachable structure such that it is convenient for cleaning so as to ensure the physiological monitoring sensor strip the cleanness and sanitation. Certainly, the physiological monitoring sensor strip described above may be without the protection layer 15.

In this embodiment, the first electrode 10 is a single-side adhesive conductive tape, and is attached to the first macromolecule polymer insulation layer 11 with an adhesive-side thereof. In addition, an electrode material (such as indium tin oxide, graphene, silver nanowire film, metal or alloy) may also be directly disposed on the first macromolecule polymer insulation layer 11 to form the first electrode 10 by means of a coating or sputtering process, which will not be limited herein.

The first electrode 10 is connected to the first extraction electrode 16 by a riveting way which can be customized according to customer requirement without being limited herein. In specific, a first end of the first extraction electrode 16 is directly riveted on the conductive tape used as the first electrode 10. The electric conduction shielding layer 14 is connected to the second extraction electrode 17 also by the riveting way which is not limited herein. In specific, a first end of the second extraction electrode 17 is directly riveted on the conductive tape used as the electric conduction shielding layer 14.

In this embodiment, the second extraction electrode 17 is a grounding electrode, that is, the electric conduction shielding layer 14 is grounded. Varying degrees of triboelectric is generated between the first macromolecule polymer insulation layer 11 and the second macromolecule polymer insulation layer 12 when the physiological monitoring sensor strip is subjected to a pressure caused by, such as weak breath, heartbeat, turning over, leaving the bed, snoring and so on during sleeping, such that the first electrode 10 induces corresponding charges, and thus varying degrees of potential differences are present between the first electrode 10 and the electric conduction shielding layer 14, since the electric conduction shielding layer 14 is grounded to be zero potential. As a result, physiological electrical signals with different strength are output between the first extraction electrode 16 and the second extraction electrode 17. The second extraction electrode 17 is grounded (i.e. the electric conduction shielding layer 14 is grounded), by which the electric conduction shielding layer 14 is not only used as an output electrode of the physiological monitoring sensor strip described above, but also as a shielding layer with a better shielding effect after being grounded.

FIG. 2 is a schematic view showing a cross-sectional structure of a physiological monitoring sensor strip according to embodiment 2 of the present disclosure. In specific, FIG. 2 shows a cross-sectional schematic of respective layered structure inside the physiological monitoring sensor strip. As shown in FIG. 2, a difference between the structure of the physiological monitoring sensor strip according to this embodiment and that of the physiological monitoring sensor strip in FIG. 1 lies in that, the insulation layer 18 does not fully wrap the inner triboelectric generator construction composed of the first electrode 10, the first macromolecule polymer insulation layer 11, and the second macromolecule polymer insulation layer 12, such that the physiological monitoring sensor strip according to this embodiment forms a partially-wrapped structure. In specific, the insulation layer wraps fully the first electrode 10 and the first macromolecule polymer insulation layer 11, but partially the second macromolecule polymer insulation layer 12, such that a partial region of the second macromolecule polymer insulation layer 12 is in contact with the electric conduction shielding layer 14.

The electric conduction shielding layer 14 is a single-side adhesive conductive tape, fully wraps an outer surface of the insulation layer 18, and is in contact with the partial region of the second macromolecule polymer insulation layer 12. In the case that the insulation layer 18 is a single-side adhesive insulation tape, then its adhesive-side is attached to the first electrode 10, to the first macromolecule polymer insulation layer 11, and partially to the second macromolecule polymer insulation layer 12, an adhesive-side of the electric conduction shielding layer 14 is attached to the outer surface of the insulation layer 18 and a partial region of the second macromolecule polymer insulation layer 12 unwrapped by the insulation layer 18 so as to form a sealed-structure. In the case that the insulation layer 18 is a double-side adhesive insulation tape, is specific, a first adhesive-side of the insulation layer 18 (i.e. an inter surface of the insulation layer 18) is attached to the first electrode 10, to the first macromolecule polymer insulation layer 11, and partially to the second macromolecule polymer insulation layer 12, the adhesive-side of the electric conduction shielding layer 14 is attached to a second adhesive-side of the insulation layer 18 (i.e. the outer surface of the insulation layer 18) and the partial region of the second macromolecule polymer insulation layer 12 unwrapped by the insulation layer 18 so as to form the sealed-structure.

The sealed-structure formed by the second macromolecule polymer insulation layer 12, the insulation layer 18, and the electric conduction shielding layer 14, on the one hand, plays functions of waterproof and moistureproof, and thus omits a process of moistureproof treatment on outermost surface, on the other hand, achieves package of the first electrode 10, the first macromolecule polymer insulation layer 11, and the second macromolecule polymer insulation layer 12, and thus avoids the triboelectric electricity generation of the first macromolecule polymer insulation layer 11 and the second macromolecule polymer insulation layer 12 from being affected by the external environmental factors.

In addition to the above differences, the specific arrangement about other respective layers in this embodiment can make reference to the description to embodiment 1, which will not be elaborated here.

FIG. 3 is a schematic view showing a cross-sectional structure of a physiological monitoring sensor strip according to embodiment 3 of the present disclosure. In specific, FIG. 3 shows a cross-sectional schematic of respective layered structure inside the physiological monitoring sensor strip. As shown in FIG. 3, a difference between the structure of the physiological monitoring sensor strip according to this embodiment and that of the physiological monitoring sensor strip in FIG. 1 lies in that, the second triboelectric layer is a second electrode layer 22, the first macromolecule polymer insulation layer 11 provided with the first electrode 10 on the first surface thereof and the second electrode layer 22 are in a stacked arrangement, and a triboelectric interface is formed between a second surface of the first macromolecule polymer insulation layer 11 not provided with the first electrode 10 thereon and the second electrode layer 22. The physiological monitoring sensor strip shown in FIG. 3 also is of a fully-wrapped structure.

In FIG. 3, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the first electrode, and the second extraction electrode is connected to the second electrode layer; or the first extraction electrode is connected to the second electrode layer, and the second extraction electrode is connected to the electric conduction shielding layer. When the second extraction electrode is connected to the electric conduction shielding layer, the second extraction electrode is grounded. The first extraction electrode and the second extraction electrode are connected to corresponding layers by the riveting way.

In this embodiment, when the physiological monitoring sensor strip is subjected to a pressure, varying degrees of triboelectric is generated between the first macromolecule polymer insulation layer and the second electrode layer, such that varying degrees of potential differences are generated between the first electrode and the second electrode layer; and varying degrees of potential differences are generated between the first electrode and the electric conduction shielding layer and between the second electrode layer and the electric conduction shielding layer when the electric conduction shielding layer is grounded to be zero potential, thus physiological electrical signals with different strength are output between the first extraction electrode and the second extraction electrode which are connected to corresponding layers according to the connection ways described above.

In the present embodiment, the second macromolecule polymer insulation layer in embodiment 1 is substituted by the second electrode layer, in addition to the above differences, the specific arrangement about other respective layers in this embodiment can make reference to the description to embodiment 1, which will not be elaborated here.

FIG. 4 is a schematic view showing a cross-sectional structure of a physiological monitoring sensor strip according to embodiment 4 of the present disclosure. In specific, FIG. 4 shows a cross-sectional schematic of respective layered structure inside the physiological monitoring sensor strip. As shown in FIG. 4, a difference between the structure of the physiological monitoring sensor strip according to this embodiment and that of the physiological monitoring sensor strip in FIG. 4 lies in that, the second triboelectric layer is the second electrode layer 22, the first macromolecule polymer insulation layer 11 provided with the first electrode 10 on the first surface thereof and the second electrode layer 22 are in a stacked arrangement, and a triboelectric interface is formed between a second surface of the first macromolecule polymer insulation layer 11 not provided with the first electrode 10 thereon and the second electrode layer 22. The insulation layer 18 fully wraps the first electrode 10 and the first macromolecule polymer insulation layer 11, but partially wraps the second macromolecule polymer insulation layer 12, such that a partial region of the second electrode layer 22 is in contact with the electric conduction shielding layer 14. The physiological monitoring sensor strip shown in FIG. 4 is of a partially-wrapped structure.

In FIG. 4, the first extraction electrode 16 is connected to the first electrode 10, and the second extraction electrode 17 is connected to the electric conduction shielding layer 14. As a partial region of the second electrode layer 22 is in contact with the electric conduction shielding layer 14, the second extraction electrode may also be connected to the second electrode layer. The first extraction electrode and the second extraction electrode are connected to corresponding layers by the riveting way, in which the first extraction electrode may be directly riveted on the conductive tape used as the first electrode, and the second extraction electrode may be riveted on the conductive tape used as the electric conduction shielding layer, on the conductive tape used as the second electrode layer, or on a composite layer formed by adhering the electric conduction shielding layer with the second electrode layer.

In this embodiment, when the physiological monitoring sensor strip is subjected to a pressure, varying degrees of triboelectric is generated between the first macromolecule polymer insulation layer and the second electrode layer, such that varying degrees of potential differences are generated between the first electrode and the second electrode layer; and varying degrees of potential differences are present between the first electrode and the electric conduction shielding layer since a partial region of the second electrode layer is in contact with the electric conduction shielding layer. As a result, physiological electrical signals with different strength are output between the first extraction electrode and the second extraction electrode which are connected to the corresponding layers according to the connection ways described above.

In the present embodiment, the second macromolecule polymer insulation layer in embodiment 2 is substituted by the second electrode layer, in addition to the above differences, the specific arrangement about other respective layers in this embodiment can make reference to the description to embodiment 2, which will not be elaborated here.

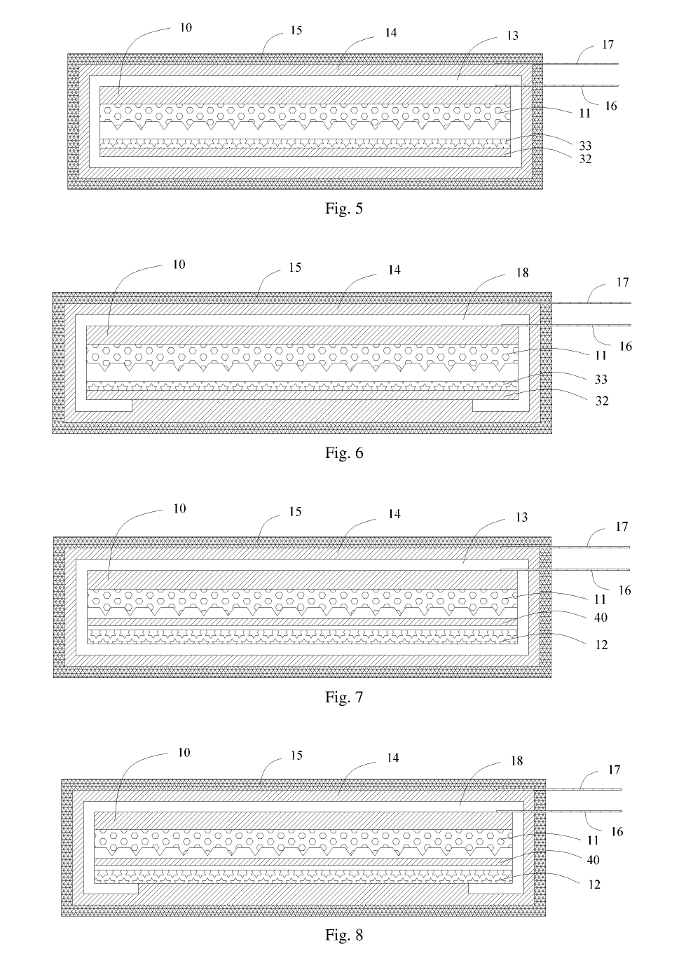

FIG. 5 is a schematic view showing a cross-sectional structure of a physiological monitoring sensor strip according to embodiment 5 of the present disclosure. In specific, FIG. 5 shows a cross-sectional schematic of respective layered structure inside the physiological monitoring sensor strip. As shown in FIG. 5, a difference between the structure of the physiological monitoring sensor strip according to this embodiment and that of the physiological monitoring sensor strip in FIG. 1 lies in that, the second triboelectric layer is a second macromolecule polymer insulation layer 33 provided with a second electrode 32 on a first surface thereof, the first macromolecule polymer insulation layer 11 provided with the first electrode 10 on the first surface thereof and the second macromolecule polymer insulation layer 33 provided with the second electrode 32 on the first surface thereof are in a stacked arrangement, and a triboelectric interface is formed between a second surface of the first macromolecule polymer insulation layer 11 not provided with the first electrode 10 thereon and a second surface of the second macromolecule polymer insulation layer 33 not provided with the second electrode 3 2 thereon. The physiological monitoring sensor strip shown in FIG. 5 is of a fully-wrapped structure.

In FIG. 5, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the first electrode, and the second extraction electrode is connected to the second electrode; or the first extraction electrode is connected to the second electrode, and the second extraction electrode is connected to the electric conduction shielding layer. When the second extraction electrode is connected to the electric conduction shielding layer, the second extraction electrode is grounded. The first extraction electrode and the second extraction electrode are connected to corresponding layers by the riveting way.

In this embodiment, when the physiological monitoring sensor strip is subjected to a pressure, varying degrees of triboelectric is generated between the first macromolecule polymer insulation layer and the second macromolecule polymer insulation layer, such that varying degrees of potential differences are generated between the first electrode and the second electrode; and varying degrees of potential differences are generated between the first electrode and the electric conduction shielding layer and between the second electrode and the electric conduction shielding layer when the electric conduction shielding layer is grounded to be zero potential, thus physiological electrical signals with different strength are output between the first extraction electrode and the second extraction electrode which are connected to the corresponding layers according to the connection ways described above.

In the present embodiment, the second macromolecule polymer insulation layer in embodiment 1 is substituted by the second macromolecule polymer insulation layer provided with the second electrode on the first surface thereof, in addition to the above differences, the specific arrangement about other respective layers in this embodiment can make reference to the description to embodiment 1, which will not be elaborated here.

FIG. 6 is a schematic view showing a cross-sectional structure of a physiological monitoring sensor strip according to embodiment 6 of the present disclosure. In specific, FIG. 6 shows a cross-sectional schematic of respective layered structure inside the physiological monitoring sensor strip. As shown in FIG. 6, a difference between the structure of the physiological monitoring sensor strip according to this embodiment and that of the physiological monitoring sensor strip in FIG. 2 lies in that, the second triboelectric layer is the second macromolecule polymer insulation layer 33 provided with the second electrode 32 on the first surface thereof, the first macromolecule polymer insulation layer 11 provided with the first electrode 10 on the first surface thereof and the second macromolecule polymer insulation layer 33 provided with the second electrode 32 on the first surface thereof are in a stacked arrangement, and a triboelectric interface is formed between a second surface of the first macromolecule polymer insulation layer 11 not provided with the first electrode 10 thereon and a second surface of the second macromolecule polymer insulation layer 33 not provided with the second electrode 3 2 thereon. The insulation layer 18 fully wraps the first electrode 10, the first macromolecule polymer insulation layer 11, and the second macromolecule polymer insulation layer 33, but partially wraps the second electrode 32, such that a partial region of the second electrode 32 is in contact with the electric conduction shielding layer 14. The physiological monitoring sensor strip shown in FIG. 6 is of a partially-wrapped structure.

In FIG. 6, the first extraction electrode 16 is connected to the first electrode 10, and the second extraction electrode 17 is connected to the electric conduction shielding layer 14. As a partial region of the second electrode 32 is in contact with the electric conduction shielding layer 14, the second extraction electrode may also be connected to the second electrode. The first extraction electrode and the second extraction electrode are connected to the corresponding layers by the riveting way, in which the first extraction electrode may be directly riveted on the conductive tape used as the first electrode, and the second extraction electrode may be riveted on the conductive tape used as the electric conduction shielding layer, on the second electrode of the second macromolecule polymer insulation layer, or on a composite layer formed by adhering the second electrode with the electric conduction shielding layer.

In this embodiment, when the physiological monitoring sensor strip is subjected to a pressure, varying degrees of triboelectric is generated between the first macromolecule polymer insulation layer and the second macromolecule polymer insulation layer, such that varying degrees of potential differences are generated between the first electrode and the second electrode; and varying degrees of potential differences are present between the first electrode and the electric conduction shielding layer since a partial region of the second electrode is in contact with the electric conduction shielding layer. As a result, physiological electrical signals with different strength are output between the first extraction electrode and the second extraction electrode which are connected to the corresponding layers according to the connection ways described above.

In the present embodiment, the second macromolecule polymer insulation layer in embodiment 2 is substituted by the second macromolecule polymer insulation layer provided with the second electrode on the first surface thereof, in addition to the above differences, the specific arrangement about other respective layers in this embodiment can make reference to the description to embodiment 2, which will not be elaborated here.

FIG. 7 is a schematic view showing a cross-sectional structure of a physiological monitoring sensor strip according to embodiment 7 of the present disclosure. In specific, FIG. 7 shows a cross-sectional schematic of respective layered structure inside the physiological monitoring sensor strip. As shown in FIG. 7, a difference between the structure of the physiological monitoring sensor strip according to this embodiment and that of the physiological monitoring sensor strip in FIG. 1 lies in that, in addition to the second macromolecule polymer insulation layer 12, the second triboelectric layer further includes an intermediate electrode layer 40, the first macromolecule polymer insulation layer 11 provided with the first electrode 10 on the first surface thereof, the intermediate electrode layer 40 and the second macromolecule polymer insulation layer 12 are in a stacked arrangement, and a triboelectric interface is formed between the second surface of the first macromolecule polymer insulation layer 11 not provided with the first electrode 10 thereon and the intermediate electrode layer 40 and/or between the second macromolecule polymer insulation layer 12 and the intermediate electrode layer 40. The physiological monitoring sensor strip shown in FIG. 7 is of a fully-wrapped structure.

In FIG. 7, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the intermediate electrode layer, and the second extraction electrode is connected to the electric conduction shielding layer; or the first extraction electrode is connected to the first electrode, and the second extraction electrode is connected to the intermediate electrode layer. When the second extraction electrode is connected to the electric conduction shielding layer, the second extraction electrode is grounded. The first extraction electrode and the second extraction electrode are connected to the corresponding layers by the riveting way.

In this embodiment, when the physiological monitoring sensor strip is subjected to a pressure, varying degrees of triboelectric is generated between the first macromolecule polymer insulation layer and the intermediate electrode layer and/or between the second macromolecule polymer insulation layer and the intermediate electrode layer, such that varying degrees of potential differences are generated between the first electrode and the intermediate electrode layer; and varying degrees of potential differences are generated between the first electrode and the electric conduction shielding layer and between the intermediate electrode layer and the electric conduction shielding layer when the electric conduction shielding layer is grounded to be zero potential. As a result, physiological electrical signals with different strength are output between the first extraction electrode and the second extraction electrode which are connected to the corresponding layers according to the connection ways described above.

In the present embodiment, the second macromolecule polymer insulation layer in embodiment 1 is substituted by the intermediate electrode layer and the second macromolecule polymer insulation layer in the stacked arrangement, in addition to the above differences, the specific arrangement about other respective layers in this embodiment can make reference to the description to embodiment 1, which will not be elaborated here.

FIG. 8 is a schematic view showing a cross-sectional structure of a physiological monitoring sensor strip according to embodiment 8 of the present disclosure. In specific, FIG. 8 shows a cross-sectional schematic of respective layered structure inside the physiological monitoring sensor strip. As shown in FIG. 8, a difference between the structure of the physiological monitoring sensor strip according to this embodiment and that of the physiological monitoring sensor strip in FIG. 2 lies in that, in addition to the second macromolecule polymer insulation layer 12, the second triboelectric layer further includes an intermediate electrode layer 40, the first macromolecule polymer insulation layer 11 provided with the first electrode 10 on the first surface thereof, the intermediate electrode layer 40 and the second macromolecule polymer insulation layer 12 are in a stacked arrangement, and a triboelectric interface is formed between the second surface of the first macromolecule polymer insulation layer 11 not provided with the first electrode 10 thereon and the intermediate electrode layer 40 and/or between the second macromolecule polymer insulation layer 12 and the intermediate electrode layer 40. The insulation layer 18 fully wraps the first electrode 10, the first macromolecule polymer insulation layer 11 and the intermediate electrode layer 40, but partially wraps the second macromolecule polymer insulation layer 12, such that a partial region of the second macromolecule polymer insulation layer 12 is in contact with the electric conduction shielding layer 14. The physiological monitoring sensor strip shown in FIG. 8 is of a partially-wrapped structure.

In FIG. 8, the second macromolecule polymer insulation layer 12 is in contact with the electric conduction shielding layer 14, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the intermediate electrode layer, and the second extraction electrode is connected to the electric conduction shielding layer; or the first extraction electrode is connected to the first electrode, and the second extraction electrode is connected to the intermediate electrode layer. When the second extraction electrode is connected to the electric conduction shielding layer, the second extraction electrode is grounded. The first extraction electrode and the second extraction electrode are connected to the corresponding layers by the riveting way.

In this embodiment, when the physiological monitoring sensor strip is subjected to a pressure, varying degrees of triboelectric is generated between the first macromolecule polymer insulation layer and the intermediate electrode layer and/or between the second macromolecule polymer insulation layer and the intermediate electrode layer, such that varying degrees of potential differences are generated between the first electrode and the intermediate electrode layer; and varying degrees of potential differences are generated between the first electrode and the electric conduction shielding layer and between the intermediate electrode layer and the electric conduction shielding layer when the electric conduction shielding layer is grounded to be zero potential. As a result, physiological electrical signals with different strength are output between the first extraction electrode and the second extraction electrode which are connected to the corresponding layers according to the connection ways described above.

In the present embodiment, the second macromolecule polymer insulation layer in embodiment 2 is substituted by the intermediate electrode layer and the second macromolecule polymer insulation layer in the stacked arrangement, in addition to the above differences, the specific arrangement about other respective layers in this embodiment can make reference to the description to embodiment 2, which will not be elaborated here.

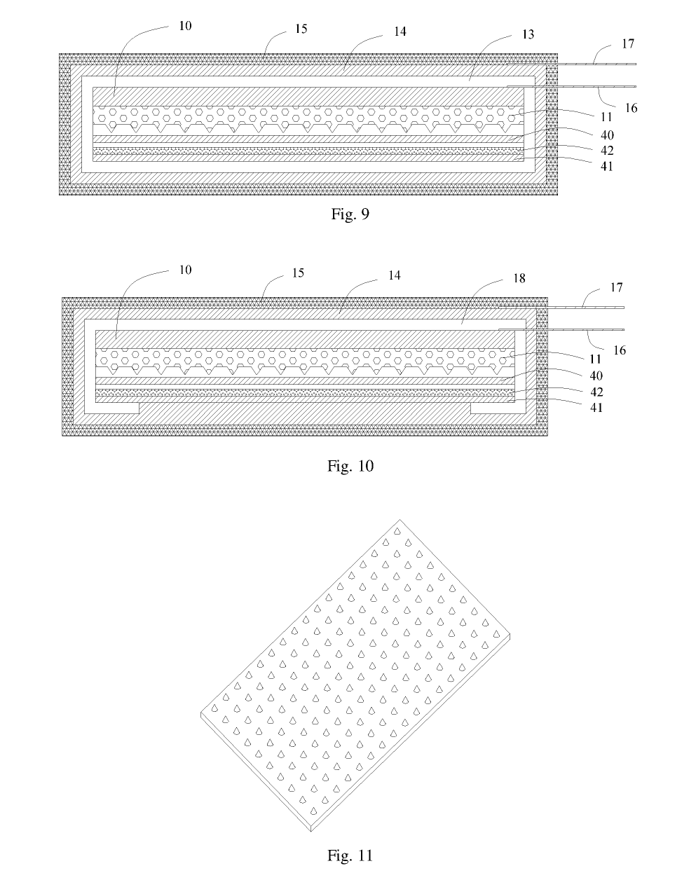

FIG. 9 is a schematic view showing a cross-sectional structure of a physiological monitoring sensor strip according to embodiment 9 of the present disclosure. In specific, FIG. 9 shows a cross-sectional schematic of respective layered structure inside the physiological monitoring sensor strip. As shown in FIG. 9, a difference between the structure of the physiological monitoring sensor strip according to this embodiment and that of the physiological monitoring sensor strip in FIG. 1 lies in that, the second triboelectric layer includes an intermediate electrode layer 40 and a second macromolecule polymer insulation layer 42 provided with a second electrode 41 on a first surface thereof; the first macromolecule polymer insulation layer 11 provided with the first electrode 10 on the first surface thereof, the intermediate electrode layer 40 and the second macromolecule polymer insulation layer 42 provided with the second electrode 41 on the first surface thereof are in a stacked arrangement in turn; and a triboelectric interface is formed between the second surface of the first macromolecule polymer insulation layer 11 not provided with the first electrode 10 thereon and the intermediate electrode layer 40 and/or between a second surface of the second macromolecule polymer insulation layer 42 not provided with the second electrode 41 thereon and the intermediate electrode layer 40. The physiological monitoring sensor strip shown in FIG. 9 is of a fully-wrapped structure.

In FIG. 9, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the first electrode, and the second extraction electrode is connected to the intermediate electrode layer or the second electrode; or the first extraction electrode is connected to the intermediate electrode layer, and the second extraction electrode is connected to the second electrode; or the first extraction electrode is connected to the intermediate electrode layer or the second electrode, and the second extraction electrode is connected to the electric conduction shielding layer. When the second extraction electrode is connected to the electric conduction shielding layer, the second extraction electrode is grounded. The first extraction electrode and the second extraction electrode are connected to the corresponding layers by the riveting way.

In this embodiment, when the physiological monitoring sensor strip is subjected to a pressure, varying degrees of triboelectric is generated between the first macromolecule polymer insulation layer and the intermediate electrode layer and/or between the second macromolecule polymer insulation layer and the intermediate electrode layer, such that varying degrees of potential differences are generated between the first electrode and the intermediate electrode layer, between the second electrode and the intermediate electrode layer, and between the first electrode and the second electrode; and varying degrees of potential differences are generated between the first electrode and the electric conduction shielding layer, between the intermediate electrode layer and the electric conduction shielding layer and between the second electrode and the electric conduction shielding layer when the electric conduction shielding layer is grounded to be zero potential. As a result, physiological electrical signals with different strength are output between the first extraction electrode and the second extraction electrode which are connected to the corresponding layers according to the connection ways described above.

In this present embodiment, the second macromolecule polymer insulation layer in embodiment 1 is substituted by the intermediate electrode layer and the second macromolecule polymer insulation layer provided with the second electrode on the first surface thereof in the stacked arrangement, in addition to the above differences, the specific arrangement about other respective layers in this embodiment can make reference to the description to embodiment 1, which will not be elaborated here.

FIG. 10 is a schematic view showing a cross-sectional structure of a physiological monitoring sensor strip according to embodiment 10 of the present disclosure. In specific, FIG. 10 shows a cross-sectional schematic of respective layered structure inside the physiological monitoring sensor strip. As shown in FIG. 10, a difference between the structure of the physiological monitoring sensor strip according to this embodiment and that of the physiological monitoring sensor strip in FIG. 2 lies in that, the second triboelectric layer includes the intermediate electrode layer 40 and the second macromolecule polymer insulation layer 42 provided with the second electrode 41 on the first surface thereof; the first macromolecule polymer insulation layer 11 provided with the first electrode 10 on the first surface thereof, the intermediate electrode layer 40 and the second macromolecule polymer insulation layer 42 provided with the second electrode 41 on the first surface thereof are in the stacked arrangement in turn; and a triboelectric interface is formed between the second surface of the first macromolecule polymer insulation layer 11 not provided with the first electrode 10 thereon and the intermediate electrode layer 40 and/or between the second surface of the second macromolecule polymer insulation layer 42 not provided with the second electrode 41 thereon and the intermediate electrode layer 40. The insulation layer 18 fully wraps the first electrode 10, the first macromolecule polymer insulation layer 11, the intermediate electrode layer 40 and the second macromolecule polymer insulation layer 42, but partially wraps the second electrode 41, such that a partial region of the second electrode 41 is in contact with the electric conduction shielding layer 14. The physiological monitoring sensor strip shown in FIG. 10 is of a partially-wrapped structure.

In FIG. 10, a partial region of the first surface of the second macromolecule polymer insulation layer 42 provided with the second electrode 41 thereon is in contact with the electric conduction shielding layer 14, the first extraction electrode 16 is connected to the first electrode 10, and the second extraction electrode 17 is connected to the electric conduction shielding layer 14. The connection way in the present embodiment is not limited to this, and may also be: the first extraction electrode is connected to the first electrode, and the second extraction electrode is connected to the intermediate electrode layer; or the first extraction electrode is connected to the first electrode, and the second extraction electrode is connected to the second electrode; or the first extraction electrode is connected to the intermediate electrode layer, and the second extraction electrode is connected to the second electrode or the electric conduction shielding layer. The first extraction electrode and the second extraction electrode are connected to the corresponding layers by the riveting way.

In this embodiment, when the physiological monitoring sensor strip is subjected to a pressure, varying degrees of triboelectric is generated between the first macromolecule polymer insulation layer and the intermediate electrode layer and/or between the second macromolecule polymer insulation layer and the intermediate electrode layer, such that varying degrees of potential differences are generated between the first electrode and the intermediate electrode layer, between the second electrode and the intermediate electrode layer, and between the first electrode and the second electrode; and varying degrees of potential differences are generated between the first electrode and the electric conduction shielding layer and between the intermediate electrode layer and the electric conduction shielding layer since a partial region of the second electrode is in contact with the electric conduction shielding layer. As a result, physiological electrical signals with different strength are output between the first extraction electrode and the second extraction electrode which are connected to the corresponding layers according to the connection ways described above.

In this present embodiment, the second macromolecule polymer insulation layer in embodiment 2 is substituted by the intermediate electrode layer and the second macromolecule polymer insulation layer provided with the second electrode on the first surface thereof in the stacked arrangement, in addition to the above differences, the specific arrangement about other respective layers in this embodiment can make reference to the description to embodiment 2, which will not be elaborated here.

In each embodiment described above, the first electrode 10 may be the conductive tape, whose adhesive-side is attached to the first macromolecule polymer insulation layer. The first macromolecule polymer insulation layer may be a film made of a polymer material, such as a polydimethylsiloxane film provided with a bump array, i.e. a PDMS film. The second macromolecule polymer insulation layer may also be a film made of a polymer material which preferably differs from the material used for manufacturing the first macromolecule polymer insulation layer, such as a polyethylene terephthalate film, i.e. a PET film. The second macromolecule polymer insulation layer provided with the second electrode on the first surface thereof may be the polyethylene terephthalate film provided with aluminum on its surface, i.e. a PET/Al film. The second electrode may also be the conductive tape whose adhesive-side is attached to the PET film. The second electrode layer or the intermediate electrode layer may be the conductive tape. The insulation layer may be the single-side adhesive or double-side adhesive conductive tape, such as a PET tape. The electric conduction shielding layer may be the single-side adhesive or non-adhesive conductive tape. The protection layer may be the fabric or plastic layer, or the plastic film. In the case that the protection layer is the plastic layer, the protection layer is made of a thin plastic.

In each embodiment described above, any one of two surfaces forming the triboelectric interface may be provided with a projection whose shape is not limited in the present disclosure. For example, the second surface of the first macromolecule polymer insulation layer, facing the second macromolecule polymer insulation layer, the second electrode layer or the intermediate electrode layer, is provided with the projection. FIG. 11 is a schematic view showing the second surface of the first macromolecule polymer insulation layer of the physiological monitoring sensor strip according to some embodiments of the present disclosure. As shown in FIG. 11, the first macromolecule polymer insulation layer is a rectangular film provided with a bump array on a first surface thereof, in which a distance between an outermost bump close to a first long edge of the rectangular film and the first long edge of the rectangular film is equal to a distance between an outermost bump close to a second long edge of the rectangular film and the second long edge of the rectangular film; and a distance between an outermost bump close to a first short edge of the rectangular film and the first short edge of the rectangular film is equal to a distance between an outermost bump close to a second short edge of the rectangular film and the second short edge of the rectangular film. According to some embodiments of the present disclosure, the physiological monitoring sensor strip with such a configuration may have improved sensitivity and increased monitoring stability and reliability, and may output the physiological electrical signal more stably.

Alternatively, the distance between the outermost bump close to the first long edge of the rectangular film and the first long edge of the rectangular film or the distance between the outermost bump close to the second long edge of the rectangular film and the second long edge of the rectangular film is 0.1 to 5 mm, preferably 1 mm; and the distance between the outermost bump close to the first short edge of the rectangular film and the first short edge of the rectangular film or the distance between the outermost bump close to the second short edge of the rectangular film and the second short edge of the rectangular film is 0.1 to 5 mm, preferably 1 mm

Alternatively, the bump in the bump array is of a height of 0.01 to 5 mm, preferably 1.25 mm, and a bump space of 0.01 to 30 mm, preferably 10 mm

In a preferred embodiment, the first macromolecule polymer insulation layer is the PDMS film provided with the bump array, and the second macromolecule polymer insulation layer is the PET film. The PDMS film provided with the bump array is of a thickness ranging from 50 to 1000 μm, preferably 200 μm; and the PET film is of a thickness ranging from 30 to 500 μm, preferably 50 μm.

The physiological monitoring sensor strip according to some embodiments of the present disclosure is manufactured basing the principle of the triboelectric generator, and can be laid or inlaid within a region of the mattress (such as a spring mattress and a coconut palm mattress) where a normal monitor may be performed by the physiological monitoring sensor strip, such as a region of the mattress corresponding to the chest of human body. The physiological monitoring sensor strip not only may be used to monitor frequencies and waveforms of human breath and heartbeat, but also to simultaneously monitor action signals (such as turning over, leaving the bed and snoring) and/or time points corresponding to action signals. Take monitoring sleep as an example, when a person is in a quiet sleep, physiological electric signals (such as voltage signals) output by the physiological monitoring sensor strip may reflect the frequencies and/or waveforms of breath and heartbeat of the person in the quiet sleep; when the person turns over, leaves the bed, or snores in the sleep, the physiological monitoring sensor strip may output physiological electric signals significantly different from that output when the person is in the quiet sleep. All of those can provide an accurate and reliable data base for analyzing sleeping quality and health status of human body.

As the physiological monitoring sensor strip of the present disclosure is manufactured basing the principle of the triboelectric generator, and is made of the easy-cutting and flexible film material, it has a plurality of characteristics, such as self-power supply, high sensitivity, stable output electric signals, simple operation, adjustable size, light weight, comfort and convenient to use for a user, low cost, simple structures and manufacturing processes, and suitable for large-scale industrial manufacture. At the same time, the physiological monitoring sensor strip achieves functions of self-moistureproof and self-shielding by disposing the insulation layer and the electric conduction shielding layer inside the structure itself, which not only increases the stability of electric signals output, but also prolongs the service life.

Further, the physiological monitoring sensor strip in each embodiment described above may further include a monitoring circuit (not shown in figures). The monitoring circuit is connected to the outputting electrode of the physiological monitoring sensor strip, for collecting and processing a physiological electrical signal output by the physiological monitoring sensor strip to obtain physiological data of the individual being monitored, such as the frequencies of breath and heartbeat.

The present disclosure also provides in embodiments a physiological monitoring system, which includes the physiological monitoring sensor strip according to any one of embodiments described above and a terminal device. The terminal device is used for statistically analyzing a physiological electrical signal output by the physiological monitoring sensor strip and displaying a result of the statistical analysis. A monitor may obtain the physiological data of the individual being monitored by means of the terminal device. The terminal device may be a mobile phone or a computer, etc., which may be used by the monitor to check the physiological data of the individual being monitored, also be used to store and analyze the physiological data of the individual being monitored, and to generate a monitoring report and an improvement suggestion in a corresponding time range required by the individual being monitored.

In addition, the physiological monitoring system may also include an alarm, which will be triggered to issue alerts in forms of sound and light when the physiological data indicates that the body condition of the individual being monitored is abnormal, for example when the breath, the heartbeat pause or the physiological electric signal is abnormal, so as to contribute the monitor to access to the information and give an assist in time.

The present disclosure also provides in embodiments a physiological monitoring mattress, which includes the physiological monitoring sensor strip according to any one of embodiments described above and a mattress body, in which the physiological monitoring sensor strip is disposed inside and/or outside the mattress body.

The present disclosure further provides in embodiments a physiological monitoring system, which includes the physiological monitoring mattress described above and a terminal device. The terminal device is used for statistically analyzing a physiological electrical signal output by the physiological monitoring mattress and displaying a result of the statistical analysis.

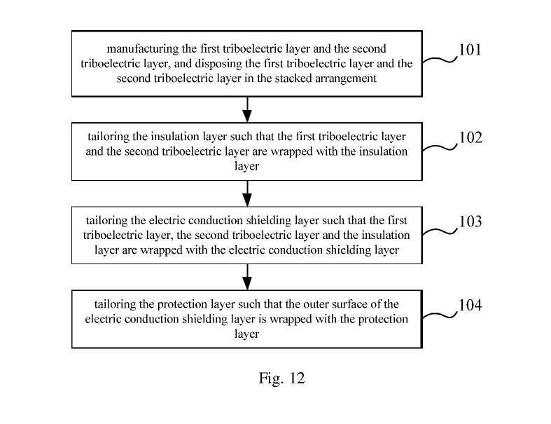

FIG. 12 is a flow chart showing a method for manufacturing a physiological monitoring sensor strip according to an embodiment of the present disclosure. As shown in FIG. 12, the method for manufacturing the physiological monitoring sensor strip includes the following steps:

Step 101: manufacturing the first triboelectric layer and the second triboelectric layer, and disposing the first triboelectric layer and the second triboelectric layer in the stacked arrangement by which a triboelectric interface is formed therebetween;

Step 102: tailoring the insulation layer such that the first triboelectric layer and the second triboelectric layer are wrapped with the insulation layer;

Step 103: tailoring the electric conduction shielding layer such that the first triboelectric layer, the second triboelectric layer and the insulation layer are wrapped with the electric conduction shielding layer; and

Step 104: tailoring the protection layer such that the outer surface of the electric conduction shielding layer is wrapped with the protection layer,

in which

the first extraction electrode and the second extraction electrode are connected to the first triboelectric layer and the second triboelectric layer, respectively, prior to tailoring the insulation layer; and exposed out when wrapped with the insulation layer, or

the first extraction electrode is connected to the first triboelectric layer or the second triboelectric layer prior to tailoring the insulation layer; and exposed out when wrapped with the insulation layer, and the second extraction electrode is connected to the electric conduction shielding layer prior to or subsequent to wrapped with the electric conduction shielding layer.

Further, the Step 101 includes: manufacturing the first macromolecule polymer insulation layer; tailoring the single-side adhesive conductive tape to obtain the first electrode; and attaching the first electrode with the first macromolecule polymer insulation layer to act as the first triboelectric layer; tailoring the conductive tape to obtain the second electrode layer to act as the second triboelectric layer; or manufacturing the second macromolecule polymer insulation layer to act as the second triboelectric layer; or disposing the second electrode on the first surface of the second macromolecule polymer insulation layer manufactured by means of a coating or sputtering process to obtain the second macromolecule polymer insulation layer provided with the second electrode on the first surface thereof to act as the second triboelectric layer.

Further, manufacturing the second triboelectric layer further includes manufacturing the intermediate electrode layer. The intermediate electrode layer and the second macromolecule polymer insulation layer or the second macromolecule polymer insulation layer provided with the second electrode on the first surface are in a stacked arrangement to act as the second triboelectric layer.

The step 102 further includes: tailoring the insulation layer such that the insulation layer is of a length larger than a sum of 2 times thicknesses of the first triboelectric layer and the second triboelectric layer plus a longer length between lengths of the first triboelectric layer and the second triboelectric layer but lower than a sum of 2 times thicknesses of the first triboelectric layer and the second triboelectric layer plus 2 times the longer length between the lengths of the first triboelectric layer and the second triboelectric layer, and of a width larger than a sum of 2 times thicknesses of the first triboelectric layer and the second triboelectric layer plus a larger width between widths of the first triboelectric layer and the second triboelectric layer but lower than a sum of 2 times thicknesses of the first triboelectric layer and the second triboelectric layer plus 2 times the larger width between the widths of the first triboelectric layer and the second triboelectric layer; and disposing the first triboelectric layer and the second triboelectric layer in a stacked arrangement in the middle of the insulation layer, and exposing out a partial region of the second triboelectric layer when wrapped with the insulation layer.

In specific, L₁, K₁ and H₁ represent the length, the width and the thicknesses of the first triboelectric layer, respectively, and L₂, K₂ and H₂ represent the length, the width and the thicknesses of the second triboelectric layer, respectively, if L₁ is larger than L₂, and K₁ is lower than K₂, then 2(H₁+H₂)+L₁< the length of the insulation layer <2(H₁+H₂)+2L₁, and 2(H₁+H₂)+K₂< the width of the insulation layer <2(H₁+H₂)+2K₂; if L₁ is lower than L₂, and K₁ is larger than K₂, then 2(H₁+H₂)+L₂< the length of the insulation layer <2(H₁+H₂)+2L₂, and 2(H₁+H₂)+K₁< the width of the insulation layer <2(H₁+H₂)+2K₁.

Alternatively, the Step 102 further includes: tailoring the insulation layer such that the insulation layer is of a length larger than or equal to a sum of thicknesses of the first triboelectric layer and the second triboelectric layer plus a longer length between lengths of the first triboelectric layer and the second triboelectric layer, and a width larger than or equal to a sum of 2 times thicknesses of the first triboelectric layer and the second triboelectric layer plus 2 times a larger width between widths of the first triboelectric layer and the second triboelectric layer; or tailoring the insulation layer such that the insulation layer is of a length larger than or equal to a sum of 2 times thicknesses of the first triboelectric layer and the second triboelectric layer plus 2 times the longer length between lengths of the first triboelectric layer and the second triboelectric layer, and a width larger than or equal to thicknesses of the first triboelectric layer and the second triboelectric layer plus a larger width between widths of the first triboelectric layer and the second triboelectric layer; and disposing the first triboelectric layer and the second triboelectric layer in a stacked arrangement in the middle of the insulation layer such that the first triboelectric layer and the second triboelectric layer are fully wrapped with the insulation layer.

In specific, L₁, K₁ and H₁ represent the length, the width and the thicknesses of the first triboelectric layer, respectively, and L₂, K₂ and H₂ represent the length, the width and the thicknesses of the second triboelectric layer, respectively, if L₁ is larger than L₂, and K₁ is lower than K₂, then the length of the insulation layer ≥H₁+H₂+L₁, and the width of the insulation layer ≥2(Hᵢ+H₂)+2K₂; if L₁ is lower than L₂, and K₁ is larger than K₂, then the length of the insulation layer ≥2(H₁+H₂)+2L₂, and the width of the insulation layer ≥H₁+H₂+K₁.

Further, in specific, the first extraction electrode and the second extraction electrode are connected, by the riveting way, to the first triboelectric layer and the second triboelectric layer, respectively, or the first extraction electrode is connected, by the riveting way, to the first triboelectric layer or the second triboelectric layer, and the second extraction electrode is connected, by the riveting way, to the electric conduction shielding layer.

Further, manufacturing the first macromolecule polymer insulation layer further includes: manufacturing a macromolecule polymer film provided with a bump array on a first surface thereof; and tailoring the macromolecule polymer film so as to obtain a rectangular film, such that a distance between an outermost bump close to a first long edge of the rectangular film and the first long edge of the rectangular film is equal to a distance between an outermost bump close to a second long edge of the rectangular film and the second long edge of the rectangular film; and a distance between an outermost bump close to a first short edge of the rectangular film and the first short edge of the rectangular film is equal to a distance between an outermost bump close to a second short edge of the rectangular film and the second short edge of the rectangular film.

Two embodiments involving in the method for manufacturing the physiological monitoring sensor strip will be described in detail below with reference to examples in which the first triboelectric layer is the PDMS film with the bump array and the second triboelectric layer is the PET/Al film.

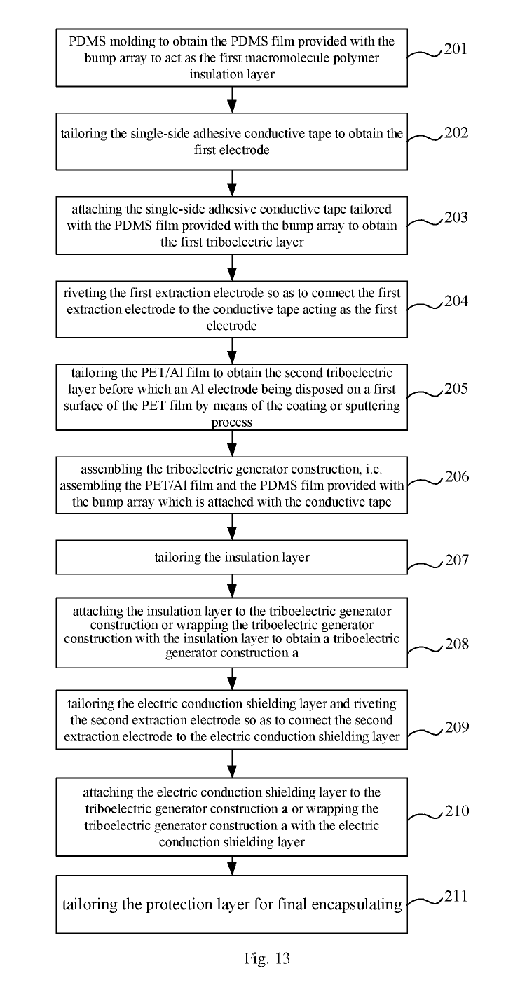

FIG. 13 is a flow chart showing a method for manufacturing a physiological monitoring sensor strip according to an embodiment of the present disclosure. As shown in FIG. 13, the method for manufacturing the physiological monitoring sensor strip includes the following steps:

Step 201: PDMS molding to obtain the PDMS film provided with the bump array to act as the first macromolecule polymer insulation layer;

Step 202: tailoring the single-side adhesive conductive tape to obtain the first electrode;

Step 203: attaching the single-side adhesive conductive tape tailored with the PDMS film provided with the bump array, i.e. attaching the first electrode with the first macromolecule polymer insulation layer, to obtain the first triboelectric layer, in specific, an adhesive-side of the conductive tape is attached to a second surface of the PDMS film not provided with the bump array thereon;

Step 204: riveting the first extraction electrode so as to connect the first extraction electrode to the conductive tape acting as the first electrode;

Step 205: tailoring the PET/Al film to obtain the second triboelectric layer before which an Al electrode is disposed on a first surface of the PET film by means of the coating or sputtering process;

Step 206: assembling a triboelectric generator construction, i.e. assembling the PET/Al film and the PDMS film provided with the bump array which is attached with the conductive tape;

Step 207: tailoring the insulation layer;

Step 208: attaching the insulation layer to the triboelectric generator construction or wrapping the triboelectric generator construction with the insulation layer to obtain a triboelectric generator construction a: in case of attaching the insulation layer to the triboelectric generator construction, in specific, choosing two pieces of insulation layers with the same size and each having a length larger than or equal to a sum of two times thicknesses of the first electrode, PET/Al film and PDMS film provided with the bump array plus a length of the PDMS film provided with the bump array, and a width larger than or equal to a sum of two times thicknesses of the first electrode, PET/Al film and PDMS film provided with the bump array plus a width of the PDMS film provided with the bump array, disposing the triboelectric generator construction between the two pieces of insulation layers, and attaching the two pieces of insulation layers to the triboelectric generator construction to be sealed and exposing the first extraction electrode at the same time, to obtain the triboelectric generator construction a; in case of wrapping the triboelectric generator construction with the insulation layer, in specific, choosing a piece of insulation layer having a length larger than or equal to a sum of thicknesses of the first electrode, the PET/Al film and the PDMS film provided with the bump array plus the length of the PDMS film provided with the bump array, and a width larger than or equal to a sum of two times thicknesses of the first electrode, the PET/Al film and the PDMS film provided with the bump array plus two times the width of the PDMS film provided with the bump array, disposing the triboelectric generator construction in the middle of the insulation layer, and wrapping the triboelectric generator construction to be sealed with the insulation layer and exposing the first extraction electrode at the same time to obtain the triboelectric generator construction a; or choosing a piece of insulation layer having a length larger than or equal to a sum of two times thicknesses of the first electrode, the PET/Al film and the PDMS film provided with the bump array plus two times the length of the PDMS film provided with the bump array, and a width larger than or equal to a sum of thicknesses of the first electrode, the PET/Al film and the PDMS film provided with the bump array plus the width of the PDMS film provided with the bump array, disposing the triboelectric generator construction in the middle of the insulation layer, and wrapping the triboelectric generator construction to be sealed with the insulation layer and exposing the first extraction electrode at the same time to obtain the triboelectric generator construction a;

Step 209: tailoring the electric conduction shielding layer and riveting the second extraction electrode so as to connect the second extraction electrode to the electric conduction shielding layer;

Step 210: attaching the electric conduction shielding layer to the triboelectric generator construction a or wrapping the triboelectric generator construction a obtained in the Step 208 with the electric conduction shielding layer to obtain a triboelectric generator construction b: in case of attaching the electric conduction shielding layer to the triboelectric generator construction a, in specific, choosing two pieces of electric conduction shielding layers with the same size and each having a length larger than or equal to a sum of two times a thickness plus a length of the triboelectric generator construction a obtained in the Step 208, and a width larger than or equal to a sum of two times the thickness plus a width of the triboelectric generator construction a obtained in the Step 208, disposing the triboelectric generator construction a obtained in the Step 208 between the two pieces of electric conduction shielding layers, and attaching the two pieces of electric conduction shielding layers to the triboelectric generator construction a obtained in the Step 208 to be sealed and exposing the first extraction electrode at the same time, to obtain the triboelectric generator construction b; in case of wrapping the triboelectric generator construction a obtained in the Step 208 with the electric conduction shielding layer, in specific, choosing a piece of electric conduction shielding layer having a length larger than or equal to a sum of the thickness plus the length of the triboelectric generator construction a obtained in the Step 208, and a width larger than or equal to a sum of two times the thickness plus two times the width of the triboelectric generator construction a obtained in the Step 208, disposing the triboelectric electricity generator construction a obtained in the Step 208 in the middle of the electric conduction shielding layer, and wrapping the triboelectric generator construction a obtained in the Step 208 to be sealed with the electric conduction shielding layer and exposing the first extraction electrode at the same time to obtain the triboelectric generator construction b; or choosing a piece of electric conduction shielding layer having a length larger than or equal to a sum of two times the thickness plus two times the length of the triboelectric generator construction a obtained in the Step 208, and a width larger than or equal to a sum of the thickness plus the width of the triboelectric generator construction a obtained in the Step 208, disposing the triboelectric generator construction a obtained in the Step 208 in the middle of the electric conduction shielding layer, and wrapping the triboelectric generator construction a obtained in the Step 208 to be sealed with the electric conduction shielding layer and exposing the first extraction electrode at the same time to obtain the triboelectric generator construction b; and

Step 211: tailoring the protection layer, and encapsulating the triboelectric electricity generator construction b with the protection layer lastly.

It should be noted that, the first electrode, the PET/Al film and the PDMS film provided with the bump array in this embodiment have the same length and the same width.

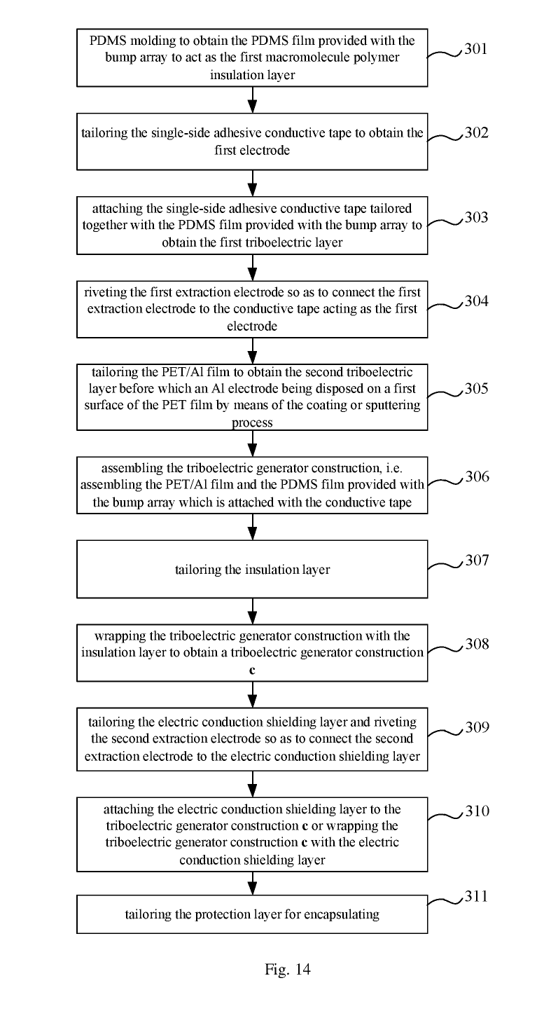

FIG. 14 is a flow chart showing a method for manufacturing a physiological monitoring sensor strip according to an embodiment of the present disclosure. As shown in FIG. 14, the method for manufacturing the physiological monitoring sensor strip includes the following steps:

Step 301: PDMS molding to obtain the PDMS film provided with the bump array to act as the first macromolecule polymer insulation layer; Step 302: tailoring the single-side adhesive conductive tape to obtain the first electrode;

Step 303: attaching the single-side adhesive conductive tape tailored with the PDMS film provided with the bump array, i.e. attaching the first electrode with the first macromolecule polymer insulation layer, to obtain the first triboelectric layer, in specific, an adhesive-side of the conductive tape being attached to a second surface of the PDMS film not provided with the bump array thereon;

Step 304: riveting the first extraction electrode so as to connect the first extraction electrode to the conductive tape acting as the first electrode;

Step 305: tailoring the PET/Al film to obtain the second triboelectric layer before which an Al electrode being disposed on a first surface of the PET film by means of the coating or sputtering process;

Step 306: assembling a triboelectric generator construction, i.e. assembling the PET/Al film and the PDMS film provided with the bump array which is attached with the conductive tape;

Step 307: tailoring the insulation layer;

Step 308: wrapping the triboelectric generator construction with the insulation layer to obtain a triboelectric generator construction c: choosing a piece of insulation layer having a length larger than a sum of two times thicknesses of the first electrode, the PET/Al film and the PDMS film provided with the bump array plus the length of the PDMS film provided with the bump array but lower than a sum of two times thicknesses of the first electrode, the PET/Al film and the PDMS film provided with the bump array plus two times the length of the PDMS film provided with the bump array, and a width larger than a sum of two times thicknesses of the first electrode, the

PET/Al film and the PDMS film provided with the bump array plus the width of the PDMS film provided with the bump array but lower than a sum of two times thicknesses of the first electrode, the PET/Al film and the PDMS film provided with the bump array plus two times the width of the PDMS film provided with the bump array, disposing the triboelectric generator construction in the middle of the insulation layer, partially wrapping the triboelectric generator construction with the insulation layer to expose a partial region of second triboelectric layer, and exposing the first extraction electrode at the same time to obtain the triboelectric generator construction c;

Step 309: tailoring the electric conduction shielding layer and riveting the second extraction electrode so as to connect the second extraction electrode to the electric conduction shielding layer;;

Step 310: attaching the electric conduction shielding layer to the triboelectric generator construction c obtained in the Step 308 or wrapping the triboelectric generator construction c obtained in the Step 308 with the electric conduction shielding layer to obtain a triboelectric generator construction d: in case of attaching the electric conduction shielding layer to the triboelectric generator construction c obtained in the Step 308, in specific, choosing two pieces of electric conduction shielding layers with the same size and each having a length larger than or equal to a sum of two times a thickness plus a length of the triboelectric generator construction c obtained in the Step 308, and a width larger than or equal to a sum of two times the thickness plus a width of the triboelectric generator construction c obtained in the Step 308, disposing the triboelectric generator construction c obtained in the Step 308 between the two pieces of electric conduction shielding layers, and attaching the two pieces of electric conduction shielding layers to the triboelectric generator construction c obtained in the Step 308 to be sealed and exposing the first extraction electrode at the same time, to obtain the triboelectric generator construction d; in case of wrapping the triboelectric generator construction c obtained in the Step 308 with the electric conduction shielding layer, in specific, choosing a piece of electric conduction shielding layer having a length larger than or equal to a sum of the thickness plus the length of the triboelectric generator construction c obtained in the Step 308, and a width larger than or equal to a sum of two times the thickness plus two times the width of the triboelectric generator construction c obtained in the Step 308, disposing the triboelectric generator construction c obtained in the Step 308 in the middle of the electric conduction shielding layer, and wrapping the triboelectric generator construction c obtained in the Step 308 to be sealed with the electric conduction shielding layer and exposing the first extraction electrode at the same time to obtain the triboelectric generator construction d; or choosing a piece of electric conduction shielding layer having a length larger than or equal to a sum of two times the thickness plus two times the length of the triboelectric generator construction c obtained in the Step 308, and a width larger than or equal to a sum of the thickness plus the width of the triboelectric generator construction c obtained in the Step 308, disposing the triboelectric generator construction c obtained in the Step 308 in the middle of the electric conduction shielding layer, and wrapping the triboelectric generator construction c obtained in the Step 308 to be sealed with the electric conduction shielding layer and exposing the first extraction electrode at the same time to obtain the triboelectric generator construction d; and

Step 311: tailoring the protection layer, and encapsulating the triboelectric generator construction d with the protection layer lastly.

It should be noted that, the first electrode, the PET/Al film and the PDMS film provided with the bump array in this embodiment have the same length and the same width.

It should be understood to those skilled in the art that, sequentially description herein are explanatory, illustrative, and used to generally understand the present disclosure, which shall not be construed to limit the present disclosure.

It can be understood that all or part of the steps in the method of the above embodiments can be implemented by instructing related hardware via programs, the program may be stored in a computer readable storage medium, such as ROM/RAM, magnetic disc, optical disc, et al.

It also should be understood that, constructions shown in figures and embodiments of the present disclosure are explanatory for achieving specific logical functions. Modules shown as separate units may be or may not be separately physically present, units shown as modules may be or may not be physical modules.

Apparently, changes, alternatives, and modifications can be made by those skilled in the art without departing from spirit, principles and scope of the present disclosure, which are within the scope of claims and the equivalent thereof, and intends to be included in the present disclosure.

1. A physiological monitoring sensor strip, comprising:
a first triboelectric layer and a second triboelectric layer in a stacked arrangement, between which a triboelectric interface is formed;
an insulation layer wrapping the first triboelectric layer and the second triboelectric layer;
an electric conduction shielding layer wrapping the first triboelectric layer, the second triboelectric layer, and the insulation layer; and
a first extraction electrode and a second extraction electrode acting as outputting electrodes of the physiological monitoring sensor strip,
wherein the first extraction electrode and the second extraction electrode are connected to the first triboelectric layer and the second triboelectric layer, respectively; or the first extraction electrode is connected to the first triboelectric layer or the second triboelectric layer, and the second extraction electrode is connected to the electric conduction shielding layer.

2. The physiological monitoring sensor strip according to claim 1, further comprising: a protection layer wrapping an outer surface of the electric conduction shielding layer.

3. The physiological monitoring sensor strip according to claim 1, wherein the insulation layer wraps fully the first triboelectric layer, and partially the second triboelectric layer such that a partial region of the second triboelectric layer is in contact with the electric conduction shielding layer.

4. The physiological monitoring sensor strip according to claim 1, wherein the insulation layer wraps fully the first triboelectric layer and the second triboelectric layer.

5. The physiological monitoring sensor strip according to claim 3, wherein
the first triboelectric layer is a first macromolecule polymer insulation layer provided with a first electrode on a first surface thereof,
the second triboelectric layer is a second macromolecule polymer insulation layer, the triboelectric interface is formed between a second surface of the first macromolecule polymer insulation layer not provided with the first electrode thereon and the second macromolecule polymer insulation layer,
the first extraction electrode is connected to the first electrode, and
the second extraction electrode is connected to the electric conduction shielding layer.

6. The physiological monitoring sensor strip according to claim 3, wherein
the first triboelectric layer is a first macromolecule polymer insulation layer provided with a first electrode on a first surface thereof,
the second triboelectric layer is a second electrode layer or a second macromolecule polymer insulation layer provided with a second electrode on a first surface thereof,
the triboelectric interface is formed between a second surface of the first macromolecule polymer insulation layer not provided with the first electrode thereon and the second electrode layer or a second surface of the second macromolecule polymer insulation layer not provided with the second electrode thereon,
the first extraction electrode is connected to the first electrode,
a partial region of the second electrode layer or a partial region of the first surface of the second macromolecule polymer insulation layer provided with the second electrode thereon is in contact with the electric conduction shielding layer, and
the second extraction electrode is connected to the second electrode layer, the second electrode or the electric conduction shielding layer.

7. The physiological monitoring sensor strip according to claim 4, wherein
the first triboelectric layer is a first macromolecule polymer insulation layer provided with a first electrode on a first surface thereof,
the second triboelectric layer is a second electrode layer or a second macromolecule polymer insulation layer provided with a second electrode on a first surface thereof,
the triboelectric interface is formed between a second surface of the first macromolecule polymer insulation layer not provided with the first electrode thereon and the second electrode layer or a second surface of the second macromolecule polymer insulation layer not provided with the second electrode thereon,
the first extraction electrode is connected to the first electrode, and
the second extraction electrode is connected to the second electrode layer or the second electrode; or the first extraction electrode is connected to the first electrode, the second electrode layer or the second electrode, and the second extraction electrode is connected to the electric conduction shielding layer.

8. The physiological monitoring sensor strip according to claim 3, wherein
the first triboelectric layer is a first macromolecule polymer insulation layer provided with a first electrode on a first surface thereof,
the second triboelectric layer comprises an intermediate electrode layer and a second macromolecule polymer insulation layer in a stacked arrangement,
the triboelectric interface is formed between a second surface of the first macromolecule polymer insulation layer not provided with the first electrode thereon and the intermediate electrode layer and/or between the second macromolecule polymer insulation layer and the intermediate electrode layer,
the first extraction electrode is connected to the first electrode or the intermediate electrode layer, and the second extraction electrode is connected to the electric conduction shielding layer; or the first extraction electrode is connected to the first electrode, and the second extraction electrode is connected to the intermediate electrode layer.

9. The physiological monitoring sensor strip according to claim 3, wherein
the first triboelectric layer is a first macromolecule polymer insulation layer provided with a first electrode on a first surface thereof,
the second triboelectric layer comprises an intermediate electrode layer and a second macromolecule polymer insulation layer provided with a second electrode on a first surface thereof which are in a stacked arrangement,
the triboelectric interface is formed between a second surface of the first macromolecule polymer insulation layer not provided with the first electrode thereon and the intermediate electrode layer and/or between a second surface of the second macromolecule polymer insulation layer not provided with the second electrode thereon and the intermediate electrode layer,
the first extraction electrode is connected to the first electrode, and the second extraction electrode is connected to the intermediate electrode layer; or the first extraction electrode is connected to the first electrode or the intermediate electrode layer, a partial region of the first surface of the second macromolecule polymer insulation layer provided with the second electrode thereon is in contact with the electric conduction shielding layer, and the second extraction electrode is connected to the second electrode or the electric conduction shielding layer.

10. The physiological monitoring sensor strip according to claim 4, wherein
the first triboelectric layer is a first macromolecule polymer insulation layer provided with a first electrode on a first surface thereof,
the second triboelectric layer comprises an intermediate electrode layer and a second macromolecule polymer insulation layer provided with a second electrode on a first surface thereof which are in a stacked arrangement,
the triboelectric interface is formed between a second surface of the first macromolecule polymer insulation layer not provided with the first electrode thereon and the intermediate electrode layer and/or between a second surface of the second macromolecule polymer insulation layer not provided with the second electrode thereon and the intermediate electrode layer,
the first extraction electrode is connected to the first electrode, and the second extraction electrode is connected to the intermediate electrode layer or the second electrode; or
the first extraction electrode is connected to the intermediate electrode layer, and the second extraction electrode is connected to the second electrode; or
the first extraction electrode is connected to the first electrode, the intermediate electrode layer or the second electrode, and the second extraction electrode is connected to the electric conduction shielding layer.

11. The physiological monitoring sensor strip according to claim 5, wherein the second extraction electrode connected to the electric conduction shielding layer is a grounding electrode.

12. The physiological monitoring sensor strip according to claim 1, wherein at least one of two surfaces forming the triboelectric interface is provided with a projection.

13. The physiological monitoring sensor strip according to claim 1, wherein the first extraction electrode and the second extraction electrode are connected to the first triboelectric layer and the second triboelectric layer in a riveting way, respectively; or
the first extraction electrode is connected to the first triboelectric layer or the second triboelectric layer in a riveting way, and the second extraction electrode are connected to the electric conduction shielding layer in a riveting way.

14. The physiological monitoring sensor strip according to any one of claim 5, wherein
the first electrode and the second electrode each are a single-side adhesive conductive tape,
the first macromolecule polymer insulation layer is a polydimethylsiloxane film provided with a bump array,
the second macromolecule polymer insulation layer is a polyethylene terephthalate film,
the second electrode layer is a conductive tape,
the insulation layer is a single-side adhesive or double-side adhesive polyethylene terephthalate tape,
the electric conduction shielding layer is a single-side adhesive or non-adhesive conductive tape, and
the protection layer is a fabric or a plastic layer, or a plastic film.

15. The physiological monitoring sensor strip according to claim 14, wherein the polydimethylsiloxane film provided with the bump array is of a thickness ranging from 50 to 1000 μm, and the polyethylene terephthalate film is of a thickness ranging from 30 to 500 μm.

16. The physiological monitoring sensor strip according to claim 5, wherein the first macromolecule polymer insulation layer is a rectangular film provided with a bump array on a first surface thereof,
wherein a distance between an outermost bump close to a first long edge of the rectangular film and the first long edge of the rectangular film is equal to a distance between an outermost bump close to a second long edge of the rectangular film and the second long edge of the rectangular film; and a distance between an outermost bump close to a first short edge of the rectangular film and the first short edge of the rectangular film is equal to a distance between an outermost bump close to a second short edge of the rectangular film and the second short edge of the rectangular film.

17. The physiological monitoring sensor strip according to claim 16, wherein
the distance between the outermost bump close to the first long edge of the rectangular film and the first long edge of the rectangular film or the distance between the outermost bump close to the second long edge of the rectangular film and the second long edge of the rectangular film is 0.1 to 5 mm; and
the distance between the outermost bump close to the first short edge of the rectangular film and the first short edge of the rectangular film or the distance between the outermost bump close to the second short edge of the rectangular film and the second short edge of the rectangular film is 0.1 to 5 mm.

18. The physiological monitoring sensor strip according to claim 16, wherein the bump in the bump array is of a height of 0.01 to 5 mm, and a bump space of 0.01 to 30 mm.

19. The physiological monitoring sensor strip according to claim 1, further comprising: a monitoring circuit, connected to the outputting electrode of the physiological monitoring sensor strip, to collect and process a physiological electrical signal output by the physiological monitoring sensor strip.

20. A physiological monitoring system comprising the physiological monitoring sensor strip according to claim 1, further comprising a terminal device, for statistically analyzing a physiological electrical signal output by the physiological monitoring sensor strip and displaying a result of the statistical analysis.

21. A physiological monitoring mattress, comprising the physiological monitoring sensor strip according to claim 1 and a mattress body, wherein the physiological monitoring sensor strip is disposed inside and/or outside the mattress body.

22. A physiological monitoring system comprising the physiological monitoring mattress according to claim 21, further comprising a terminal device for statistically analyzing a physiological electrical signal output by the physiological monitoring mattress and display a result of the statistical analysis.

23. A method for manufacturing a physiological monitoring sensor strip, comprising:
manufacturing a first triboelectric layer and a second triboelectric layer, and disposing the first triboelectric layer and the second triboelectric layer in a stacked arrangement by which a triboelectric interface is formed therebetween;
tailoring an insulation layer such that the first triboelectric layer and the second triboelectric layer are wrapped with the insulation layer; and
tailoring an electric conduction shielding layer such that the first triboelectric layer, the second triboelectric layer and the insulation layer are wrapped with the electric conduction shielding layer,
wherein
a first extraction electrode and a second extraction electrode are connected to the first triboelectric layer and the second triboelectric layer, respectively, prior to tailoring the insulation layer; and exposed out when wrapped with the insulation layer, or
the first extraction electrode is connected to the first triboelectric layer or the second triboelectric layer prior to tailoring the insulation layer; and exposed out when wrapped with the insulation layer, and the second extraction electrode is connected to the electric conduction shielding layer prior to or subsequent to wrapped with the electric conduction shielding layer.

24. The method according to claim 23, wherein subsequent to wrapping the first triboelectric layer, the second triboelectric layer and the insulation layer with the electric conduction shielding layer, the method further comprises:
tailoring a protection layer such that an outer surface of the electric conduction shielding layer is wrapped with the protection layer.

25. The method according to claim 23, wherein tailoring the insulation layer such that the first triboelectric layer and the second triboelectric layer are wrapped with the insulation layer further comprises:
tailoring the insulation layer such that the insulation layer is of a length larger than a sum of 2 times thicknesses of the first triboelectric layer and the second triboelectric layer plus a longer length between lengths of the first triboelectric layer and the second triboelectric layer but lower than a sum of 2 times thicknesses of the first triboelectric layer and the second triboelectric layer plus 2 times the longer length between the lengths of the first triboelectric layer and the second triboelectric layer, and of a width larger than a sum of 2 times thicknesses of the first triboelectric layer and the second triboelectric layer plus a larger width between widths of the first triboelectric layer and the second triboelectric layer but lower than a sum of 2 times thicknesses of the first triboelectric layer and the second triboelectric layer plus 2 times the larger width between the widths of the first triboelectric layer and the second triboelectric layer; and
disposing the first triboelectric layer and the second triboelectric layer in a stacked arrangement between the insulation layers, and exposing out a partial region of the second triboelectric layer when wrapped with the insulation layers.

26. The method according to claim 23, wherein tailoring the insulation layer such that the first triboelectric layer and the second triboelectric layer are wrapped with the insulation layer further comprises:
tailoring the insulation layer such that the insulation layer is of a length larger than or equal to a sum of thicknesses of the first triboelectric layer and the second triboelectric layer plus a longer length between lengths of the first triboelectric layer and the second triboelectric layer, and a width larger than or equal to a sum of 2 times thicknesses of the first triboelectric layer and the second triboelectric layer plus 2 times a larger width between widths of the first triboelectric layer and the second triboelectric layer, or
tailoring the insulation layer such that the insulation layer is of a length larger than or equal to a sum of 2 times thicknesses of the first triboelectric layer and the second triboelectric layer plus 2 times a longer length between lengths of the first triboelectric layer and the second triboelectric layer, and a width larger than or equal to thicknesses of the first triboelectric layer and the second triboelectric layer plus a larger width between widths of the first triboelectric layer and the second triboelectric layer; and
disposing the first triboelectric layer and the second triboelectric layer in a stacked arrangement in the middle of the insulation layer such that the first triboelectric layer and the second triboelectric layer are fully wrapped with the insulation layer.

27. The method according to claim 23, wherein manufacturing the first triboelectric layer further comprises:
manufacturing a first macromolecule polymer insulation layer;
tailoring a single-side adhesive conductive tape to obtain a first electrode; and
attaching the first electrode with the first macromolecule polymer insulation layer.

28. The method according to claim 27, wherein manufacturing the second triboelectric layer further comprises:
tailoring a conductive tape to obtain a second electrode layer; or
manufacturing a second macromolecule polymer insulation layer; or
coating or sputtering a second electrode on a first surface of the second macromolecule polymer insulation layer manufactured.

29. The method according to claim 28, wherein manufacturing the second triboelectric layer further comprises:
manufacturing an intermediate electrode layer; and
disposing the intermediate electrode layer and the second macromolecule polymer insulation layer or the second macromolecule polymer insulation layer coated or sputtered with the second electrode on the first surface in a stacked arrangement as the second triboelectric layer.

30. The method according to claim 23, wherein
the first extraction electrode and the second extraction electrode are connected, by a riveting way, to the first triboelectric layer and the second triboelectric layer, respectively, or
the first extraction electrode is connected, by a riveting way, to the first triboelectric layer or the second triboelectric layer, and
the second extraction electrode is connected, by a riveting way, to the electric conduction shielding layer.

31. The method according to claim 27, wherein manufacturing the first macromolecule polymer insulation layer further comprises:
manufacturing a macromolecule polymer film provided with a bump array on a first surface thereof; and
tailoring the macromolecule polymer film so as to obtain a rectangular film, such that a distance between an outermost bump close to a first long edge of the rectangular film and the first long edge of the rectangular film is equal to a distance between an outermost bump close to a second long edge of the rectangular film and the second long edge of the rectangular film; and a distance between an outermost bump close to a first short edge of the rectangular film and the first short edge of the rectangular film is equal to a distance between an outermost bump close to a second short edge of the rectangular film and the second short edge of the rectangular film.
